(19) United States
(12) Patent Application Publication
(10) Pub. No.: US 2005/0187184 A1
(43) Pub. Date: Aug. 25, 2005

(54) Antineoplastic combinations

(75) Inventors: James J. Gibbons JR., Westwood, NJ (US); Gary Dukart, Ambler, PA (US); Jurgen Frisch, Marburg (DE)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 11/110,050

(22) Filed: Apr. 20, 2005

Related U.S. Application Data
(63) Continuation of application No. 10/117,161, filed on Apr. 5, 2002.
(60) Provisional application No. 60/282,385, filed on Apr. 6, 2001.

(51) Int. Cl. A61K 31/7072; A61K 31/4745

(52) U.S. Cl. 514/49; 514/291

(57) ABSTRACT

This invention provides the use of a combination of CCI-779 and an antimetabolite antineoplastic agent in the treatment of neoplasms.

### CROSS-REFERENCE TO RELATED APPLICATIONS

This application is a continuation of U.S. patent application Ser. No. 10/117,161, filed Apr. 5, 2002, which claims the benefit of the priority of U.S. Patent Application No. 60/282,385, filed Apr. 6, 2001.

### BACKGROUND OF THE INVENTION

This invention relates to the use of combinations of rapamycin 42-ester with 3-hydroxy-2-(hydroxymethyl)-2-methylpropionic acid (CCI-779) and an antimetabolite antineoplastic agent in the treatment of neoplasms.

Rapamycin is a macrocyclic triene antibiotic produced by Streptomyces hygroscopicus, which was found to have antifungal activity, particularly against Candida albicans, both in vitro and in vivo [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. No. 3,929,992; and U.S. Pat. No. 3,993,749]. Additionally, rapamycin alone (U.S. Pat. No. 4,885,171) or in combination with picibanil (U.S. Pat. No. 4,401,653) has been shown to have antitumor activity.

The immunosuppressive effects of rapamycin have been disclosed in FASEB 3, 3411 (1989). Cyclosporin A and FK-506, other macrocyclic molecules, also have been shown to be effective as immunosuppressive agents, therefore useful in preventing transplant rejection [FASEB 3, 3411 (1989); FASEB 3, 5256 (1989); R. Y. Calne et al., Lancet 1183 (1978); and U.S. Pat. No. 5,100,899]. R. Martel et al. [Can. J. Physiol. Pharmacol. 55, 48 (1977)] disclosed that rapamycin is effective in the experimental allergic encephalomyelitis model, a model for multiple sclerosis; in the adjuvant arthritis model, a model for rheumatoid arthritis; and effectively inhibited the formation of IgE-like antibodies.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,0079], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 Al], ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

Rapamycin 42-ester with 3-hydroxy-2-(hydroxymethyl)-2-methylpropionic acid (CCI-779) is ester of rapamycin which has demonstrated significant inhibitory effects on tumor growth in both in vitro and in vivo models. The preparation and use of hydroxyesters of rapamycin, including CCI-779, are disclosed in U.S. Pat. No. 5,362,718.

CCI-779 exhibits cytostatic, as opposed to cytotoxic properties, and may delay the time to progression of tumors or time to tumor recurrence. CCI-779 is considered to have a mechanism of action that is similar to that of sirolimus. CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S. The mechanism of action of CCI-779 that results in the G_{1\hat{ }} S phase block is novel for an anticancer drug.

In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

In vivo studies in nude mice have demonstrated that CCI-779 has activity against human tumor xenografts of diverse histological types. Gliomas were particularly sensitive to CCI-779 and the compound was active in an orthotopic glioma model in nude mice. Growth factor (platelet-derived)-induced stimulation of a human glioblastoma cell line in vitro was markedly suppressed by CCI-779. The growth of several human pancreatic tumors in nude mice as well as one of two breast cancer lines studied in vivo also was inhibited by CCI-779.

### DESCRIPTION OF THE INVENTION

This invention provides the use of combinations of CCI-779 and an antimetabolite antineoplastic agent as antineoplastic combination chemotherapy. In particular, these combinations are useful in the treatment of renal cancer, soft tissue cancer, breast cancer, neuroendocrine tumor of the lung, cervical cancer, uterine cancer, head and neck cancer, glioma, non-small lung cell cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. This invention also provides combinations of CCI-779 and an antimetabolite antineoplastic agent for use as antineoplastic combination chemotherapy, in which the dosage of either CCI-779 or the antimetabolite antineoplastic agent or both are used in subtherapeutically effective dosages.

As used in accordance with this invention, the term “treatment” means treating a mammal having a neoplastic disease by providing said mammal an effective amount of a combination of CCI-779 and an antimetabolite antineoplastic agent with the purpose of inhibiting growth of the neoplasm in such mammal, eradication of the neoplasm, or palliation of the mammal.

As used in accordance with this invention, the term “providing,” with respect to providing the combination, means either directly administering the combination, or administering a prodrug, derivative, or analog of one or both of the components of the combination which will form an effective amount of the combination within the body.

The preparation of CCI-779 is described in U.S. Pat. No. 5,362,718, which is hereby incorporated by reference. When CCI-779 is used as an antineoplastic agent, it is projected that initial i.v. infusion dosages will be between about 0.1 and 100 mg/m² when administered on a daily dosage regimen (daily for 5 days, every 2-3 weeks), and between about 0.1 and 1000 mg/m² when administered on a once weekly dosage regimen. Oral or intravenous infusion are the preferred routes of administration, with intravenous being more preferred.

As used in accordance with this invention, the term “antimetabolite” means a substance which is structurally similar to a critical natural intermediate (metabolite) in a biochemical pathway leading to DNA or RNA synthesis which is used by the host in that pathway, but acts to inhibit the completion of that pathway (i.e., synthesis of DNA or RNA). More specifically, antimetabolites typically function by (1) competing with metabolites for the catalytic or regulatory site of a key enzyme in DNA or RNA synthesis, or (2) substitute for a metabolite that is normally incorporated into DNA or RNA, and thereby producing a DNA or RNA that cannot support replication. Major categories of antimetabolites include (1) folic acid analogs, which are inhibitors of dihydrofolate reductase (DHFR); (2) purine analogs, which mimic the natural purines (adenine or guanine) but are structurally different so thy competitively or irreversibly inhibit nuclear processing of DNA or RNA; and (3) pyrimidine analogs, which mimic the natural pyrimidines (cytosine, thymidine, and uracil) but are structurally different so thy competitively or irreversibly inhibit nuclear processing of DNA or RNA.

The following are representative examples of antimetabolites of this invention.

5-Fluorouracil (5-FU; 5-fluoro-2,4(1H,3H)-pyrimidinedione) is commercially available in a topical cream (FLUOROPLEX or EFUDEX) a topical solution (FLUOROPLEX or EFUDEX), and as an injectable containing 50 mg/mL 5-fluorouracil (ADRUCIL or flurouracil).

Floxuradine (2′-deoxy-5-fluorouridine) is commercially available as an injectable containing 500 mg/vial of floxuradine (FUDR or floxuradine).

Thioguanine (2-amino-1,7-dihydro-6-H-purine-6-thione) is commercially available in 40 mg oral tablets (thioguanine).

Cytarabine (4-amino-1-(beta)-D-arabinofuranosyl-2(1H)-pyrimidinone) is commercially available as a liposomal injectable containing 10 mg/mL cytarabine (DEPOCYT) or as a liquid injectable containing between 1 mg-1 g/vial or 20 mg/mL (cytarabine or CYTOSAR-U).

Fludarabine (9-H-Purin-6-amine,2-fluoro-9-(5-O-phosphono-(beta)-D-arabinofuranosyl) is commercially available as a liquid injectable containing 50 mg/vial (FLUDARA).

6-Mercaptopurine (1,7-dihydro-6H-purine-6-thione) is commercially available in 50 mg oral tablets (PURINETHOL).

Methotrexate (MTX; N-[4-[[(2,4-diamino-6-pteridinyl)methyl]methylamino]benzoyl]-L-glutamic acid) is commercially available as a liquid injectable containing between 2.5-25 mg/mL and 20 mg-1 g/vial (methotrexate sodium or FOLEX) and in 2.5 mg oral tablets (methotrexate sodium).

Gemcitabine (2′-deoxy-2′,2′-difluorocytidine monohydrochloride ((beta)-isomer)), is commercially available as a liquid injectable containing between 200 mg-1 g/vial (GEMZAR).

Capecitabine (5′-deoxy-5-fluoro-N-[(pentyloxy)carbonyl]-cytidine) is commercially available as a 150 or 500 mg oral tablet (XELODA).

Pentostatin ((R)-3-(2-deoxy-(beta)-D-erythro-pentofuranosyl)-3,6,7,8-tetrahydroimidazo[4,5-d][1,3]diazepin-8-ol) is commercially available as a liquid injectable containing 10 mg/vial (NIPENT).

Trimetrexate (2,4-diamino-5-methyl-6-[(3,4,5-trimethoxyanilino)methyl]quinazoline mono-D-glucuronate) is commercially available as a liquid injectable containing between 25-200 mg/vial (NEUTREXIN).

Cladribine (2-chloro-6-amino-9-(2-deoxy-(beta)-D-erythropento-furanosyl)purine) is commercially available as a liquid injectable containing 1 mg/mL (LEUSTATIN).

The following table briefly summarizes some of the recommended dosages for the antimetabolites listed above.

| Drug | Dosage | | Regimen |
|---|---|---|---|
| 5-Fluorouracil | 12 | mg/kg oral | daily for 4 days |
| | 6 | mg/kg oral | days 6, 8, 10, 12 |
| | | | no drug on days 5, 7, 9, and 11; doses cut in |
| | | | half if toxicity observed |
| | 370-600 | mg/m² i.v. | daily for 5 days, every 3-4 weeks |
| Floxuradine | 0.1-0.6 | mg/kg | daily by arterial infusion |
| (FUDR) | | | |
| Cytarabine | 50 | mg | every 14 days for 5 doses during induction |
| (DEPOCYT) | | | period; followed by every 28 days for |
| | | | maintenance |
| Cytarabine | 100 | mg/m² | daily for 7 days |
| (injectable) | 2-3 | g/m² | twice daily for 2-6 days |
| Fludarabine | 25 | mg/m² | 30 min infusion for 5 consecutive days; every |
| (FLUDARA) | | | 28 days |
| 6-Mercaptopurine | 2.5-5 | mg/kg | daily for induction |
| (PURINETHOL) | 1.5-2.5 | mg/kg | daily for maintenance |
| Methotrexate | 15-30 | mg oral | daily for 5 day course; repeated 3-5 times |
| Gemcitabine | 1000 | mg/m²/30 min | single agent: once weekly for 7 weeks, |
| (GEMZAR) | | | followed by 1 week rest, then once weekly for |
| | | | 3 out of every 4 weeks |
| | 1000-1250 | mg/m²/30 min | combination therapy: days 1, 8, 15 per 28 day |
| | | | cycle, or days 1 and 8 per 21 day cycle |
| Capecitabine | 2500 | mg/m² | daily for 2 weeks followed by 1 week rest |
| (XELODA) | | | period |
| Pentostatin | 4 | mg/m² | as bolus injection or diluted as i.v. infusion; |
| (NIPENT) | | | every other week |
| Trimetrexate | 45 | mg/m² | i.v. infusion once daily for 21 days |
| (NEUTREXIN) | | | |
| Cladribine | 0.09 | mg/kg/day | continuous infusion for 7 consecutive days |
| (LEUSTATIN) | | | |

This invention also covers the use of CCI-779 plus an antimetabolite in which a biochemical modulating agent is part of the chemotherapeutic regimen. The term “biochemical modulating agent” is well known and understood to those skilled in the art as an agent given as an adjunct to antimetabolite therapy, which serves to potentate its antineoplastic activity, as well as counteract the side effects of the antimetabolite. Leucovorin and levofolinate are typically used as biochemical modulating agents for methotrexate and 5-FU therapy.

Leucovorin (5-formyl-5,6,7,8-tetrahydrofolic acid) is commercially available as an injectable liquid containing between 5-10 mg/mL or 50-350 mg/vial (leucovorin calcium or WELLCOVORIN) and as 5-25 mg oral tablets (leucovorin calcium).

Levofolinate (pharmacologically active isomer of 5-formyltetrahydrofolic acid) is commercially available as an injectable containing 25-75 mg levofolinate (ISOVORIN) or as 2.5-7.5 mg oral tablets (ISOVORIN).

Preferred combinations of this invention include CCI-779 plus gemcitabine; CCI-779 plus 5-fluorouracil; and CCI-779 plus 5-fluorouracil plus leucovorin. It is preferred that the CCI-779 plus gemcitabine combination be used in treating pancreatic cancer and that the CCI-779 plus 5-fluorouracil combination (with or without leucovorin) be used in treating colorectal cancer.

The antineoplastic activity of the CCI-779 plus antimetabolite combination was confirmed in in vitro and in vivo standard pharmacological test procedures using combinations of CCI-779 plus gemcitabine; and CCI-779 plus 5-fluorouracil as representative combinations of this invention. The following briefly describes the procedures used and the results obtained.

Human rhabdomyosarcoma lines Rh30 and Rh1 and the human glioblastoma line SJ-GBM2 were used for in vitro combination studies with CCI-779 and antimetabolite agents. In vivo studies used a human neuroblastoma (NB1643) and human colon line GC3.

Dose response curves were determined for each of the drugs of interest. The cell lines Rh30, Rh1 and SJ-G2 were plated in six-well cluster plates at 6×10³, 5×10³ and 2.5×10⁴ cells/well respectively. After a 24 hour incubation period, drugs were added in either 10%FBS+RPMI 1640 for Rh30 and Rh1 or 15%FBS+DME for SJ-G2. After seven days exposure to drug containing media, the nuclei were released by treating the cells with a hypotonic solution followed by a detergent. The nuclei were then counted with a Coulter Counter. The results of the experiments were graphed and the IC₅₀ (drug concentration producing 50% inhibition of growth) for each drug was determined by extrapolation. Because the IC50s varied slightly from experiment to experiment, two values that bracketed the IC50 of each drug were used in the interaction studies. The point of maximum interaction between two drugs occurs when they are present in a 1:1 ratio if the isobole is of standard shape. Therefore, each of the three approximate IC₅₀ concentrations of CCI-779 was mixed in a 1:1 ratio with each of three approximated IC₅₀s of gemcitabine or 5-FU. This resulted in nine 1:1 combinations of drugs in each experiment plus three IC₅₀ concentrations for CCI-779 and the other drug. This protocol usually resulted in at least one combination for each drug containing an IC₅₀ value. The 1:1 combination of IC₅₀ concentrations for CCI-779 and each chemotherapy drug was then used to calculate additivity, synergism, or antagonism using Berenbaum's formula: x/X₅₀+y/Y₅₀,=1,<1,>1. If the three concentrations of CCI-779 tested alone didn't produce an IC that matched any of the three ICs of the other compound tested alone, all the 1:1 combinations were checked to see if their ICs fell between the appropriate ICs of drugs tested singly. If they did, the effect was considered additive.

The results obtained in the in vitro standard pharmacological test procedure showed that in no case did the combinations yield less than a 50% inhibition of growth indicating that the combinations were at least additive and produced no evidence of antagonism.

Female CBA/CaJ mice (Jackson Laboratories, Bar Harbor, Me.), 4 weeks of age, were immune-deprived by thymectomy, followed 3 weeks later by whole-body irradiation (1200 cGy) using a ¹³⁷Cs source. Mice received 3×10⁶ nucleated bone marrow cells within 6-8 h of irradiation. Tumor pieces of approximately 3 mm³ were implanted in the space of the dorsal lateral flanks of the mice to initiate tumor growth. Tumor-bearing mice were randomized into groups of seven prior to initiating therapy. Mice bearing tumors each received drug when tumors were approximately 0.20-1 cm in diameter. Tumor size was determined at 7-day intervals using digital Vernier calipers interfaced with a computer. Tumor volumes were calculated assuming tumors to be spherical using the formula [(□/6)×d³], where d is the mean diameter. CCI-779 was given on a schedule of 5 consecutive days for 2 weeks with this cycle repeated every 21 days for 3 cycles. This resulted in CCI-779 being given on days 1-5, 8-12 (cycle 1); 21-25, 28-32 (cycle 2); and 42-46, 49-53 (cycle 3). The schedule of the other chemotherapy drug for each study was as follows:
Gemcitabine on days 1, 4, 8 in cycle 1 only

The combination of CCI-779 and gemcitabine was evaluated in a human colon (GC3) mouse xenograft test procedure. In this test procedure, CCI-779 was given daily ×5 for 2 consecutive weeks every 21 days for 3 cycles and gemcitabine given on days 1, 4, and 8 in the first cycle only. The presence of CCI-779 did not enhance tumor regression seen in the first cycle with gemcitabine treatment. However, groups treated with CCI-779 were delayed in the time required to reach 2-3× the original pretreatment tumor volume (versus gemcitabine alone), indicating that there was at least an additive benefit derived from the combination treatment.

Based on the results of these standard pharmacological test procedures, combinations of CCI-779 plus an antimetabolite chemotherapeutic agent are useful as antineoplastic therapy. More particularly, these combinations useful in treating treatment of renal carcinoma, soft tissue sarcoma, breast cancer, neuroendocrine tumor of the lung, cervical cancer, uterine cancer, head and neck cancer, glioma, non-small cell lung cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. As these combinations contain at least two active antineoplastic agents, the use of such combinations also provides for the use of combinations of each of the agents in which one or both of the agents is used at subtherapeutically effective dosages, thereby lessening toxicity associated with the individual chemotherapeutic agent.

In providing chemotherapy, multiple agents having different modalities of action are typically used as part of a chemotherapy “cocktail.” It is anticipated that the combinations of this invention will be used as part of a chemotherapy cocktail that may contain one or more additional antineoplastic agents depending on the nature of the neoplasia to be treated. For example, this invention also covers the use of the CCI-779/antimetabolite combination used in conjunction with other chemotherapeutic agents, such as alkylating agents (i.e., cisplatin, carboplatin, streptazoin, melphalan, chlorambucil, carmustine, methclorethamine, lomustine, bisulfan, thiotepa, ifofamide, or cyclophosphamide); hormonal agents (i.e., estramustine, tamoxifen, toremifene, anastrozole, or letrozole); antibiotics (i.e., plicamycin, bleomycin, mitoxantrone, idarubicin, dactinomycin, mitomycin, or daunorubicin); immunomodulators (i.e., interferons, IL-2, or BCG); antimitotic agents (i.e., vinblastine, vincristine, teniposide, or vinorelbine); topoisomerase inhibitors (i.e., topotecan, irinotecan, etoposide, or doxorubicin); and other agents (i.e., hydroxyurea, trastuzumab, altretamine, retuximab, paclitaxel, docetaxel, L-asparaginase, or gemtuzumab ozogamicin).

As used in this invention, the combination regimen can be given simultaneously or can be given in a staggered regimen, with CCI-779 being given at a different time during the course of chemotherapy than the antimetabolite. This time differential may range from several minutes, hours, days, weeks, or longer between administration of the two agents. Therefore, the term combination does not necessarily mean administered at the same time or as a unitary dose, but that each of the components are administered during a desired treatment period. The agents may also be administered by different routes. For example, in the combination of CCI-779 plus an antimetabolite, it is anticipated that the CCI-779 will be administered orally or parenterally, with parenterally being preferred, while the antimetabolite may be administered parenterally, orally, or by other acceptable means. For the CCI-779 combination with gemcitabine, it is preferred that the gemcitabine be administered parenterally. For the CCI-779 combination with 5-FU and leucovorin, it is preferred that the 5-FU and leucovorin are administered parenterally. These combination can be administered daily, weekly, or even once monthly. As typical for chemotherapeutic regimens, a course of chemotherapy may be repeated several weeks later, and may follow the same timeframe for administration of the two agents, or may be modified based on patient response.

As typical with chemotherapy, dosage regimens are closely monitored by the treating physician, based on numerous factors including the severity of the disease, response to the disease, any treatment related toxicities, age, health of the patient, and other concomitant disorders or treatments.

Based on the results obtained with the CCI-779 plus antimetabolite combinations, it is projected that the initial i.v. infusion dosage of CCI-779 will be between about 0.1 and 100 mg/m², with between about 2.5 and 70 mg/m² being preferred. It is also preferred that the CCI-779 be administered by i.v., typically over a 30 minute period, and administered about once per week. The initial dosages of the antimetabolite component will depend on the component used, and will be based initially on physician experience with the agents chosen.

Based on the results obtained with the CCI-779 plus antimetabolite combinations, it is projected that for the CCI-779 plus gemcitabine combination, the initial i.v. infusion dosage of CCI-779 will be between about 0.1 and 100 mg/m², with between about 2.5 and 70 mg/m² being preferred, and the initial i.v. infusion dosage of gemcitabine will be between about 400 and 1500 mg/m², with between about 800 and 1000 mg/m² being preferred. It is initially projected that patients will receive a 30 minute i.v. infusion of CCI-779, followed immediately or preceded by a 30 minute i.v. infusion of gemcitabine on days I and 8 of a 21 day treatment cycle. After one or more treatment cycles, the dosages can be adjusted upwards or downwards depending on the results obtained and the side effects observed.

Based on the results obtained, when CCI-779 is used in combination with 5-FU and leucovorin, it is projected that the initial i.v. infusion dosage of CCI-779 will be between about 0.1 and 100 mg/m², with between about 2.5 and 70 mg/m² being preferred; the initial i.v. infusion dosage of leucovorin will be between about 50 and 500 mg/m², with about 200 mg/m² being preferred; and the initial i.v. infusion dosage of 5-FU will be between about 500 and 7500 mg/m², with between about 1000 and 5000 mg/m² being preferred. It is initially projected that the combination will be administered according to the following regimen: patients will receive a 1 hour i.v. infusion of leucovorin once weekly during each 6 week treatment cycle; immediately following each dose of leucovorin, 5-FU is administered as a 24-hour continuous i.v. infusion. CCI-779 will be administered beginning on day 8, of cycle 1, and will be given once weekly as a 30 minute i.v. infusion. Each 6 week treatment cycle is followed by a 1 week rest before beginning the next 6 week treatment cycle. After one or more treatment cycles, the dosages can be adjusted upwards or downwards depending on the results obtained and the side effects observed.

For commercially available antimetabolites, the existing dosage form can be used, with the dosages divided as need be. Alternatively, such agents or antimetabolites that are not commercially available can be formulated according to standard pharmaceutical practice. Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

1. A method of treating a neoplasm in a mammal in need thereof, which comprises providing to said mammal an effective amount of a combination comprising CCI-779 and an antimetabolite antineoplastic agent.

2. The method according to claim 1, wherein said neoplasm is selected from the group consisting of: renal cancer; soft tissue sarcoma; breast cancer; a neuroendocrine tumor of the lung; cervical cancer; uterine cancer; a head and neck cancer; glioma; non-small cell lung cancer; prostate cancer; pancreatic cancer; lymphoma; melanoma; small cell lung cancer; ovarian cancer; colon cancer; esophageal cancer; gastric cancer; leukemia; colorectal cancer; and unknown primary cancer.

3. The method according to claim 2, wherein said neoplasm is soft tissue sarcoma, colon cancer, or colorectal cancer.

4. The method according to claim 1, wherein said antineoplastic alkylating agent is gemcitabine or 5-fluorouracil.

5. The method according to claim 4, wherein said antineoplastic alkylating agent is gemcitabine.

6. The method according to claim 5, wherein the neoplasm is pancreatic cancer.

7. The method according to claim 4, wherein the combination comprises 5-fluorouracil.

8. The method according to claim 7, wherein the neoplasm is colorectal cancer.

9. The method according to claim 7, wherein the combination further comprises a biochemical modifying agent.

10. The method according to claim 9, wherein the biochemical modifying agent is leucovorin.

11. The method according to claim 6, wherein CCI-779, 5-fluorouracil, or both are provided in subtherapeutically effective amounts.

12. An antineoplastic combination which comprises an effective amount of CCI-779 and 5-fluourouracil.

13. The combination according to claim 12, further comprising leucovorin.

14. A method of treating a neoplasm in a mammal in need thereof, which comprises alternating administration of CCI-779 and an antimetabolite antineoplastic agent to said mammal.

15. The method according to claim 14, wherein said neoplasm is soft tissue sarcoma, colon cancer, or colorectal cancer.

16. The method according to claim 14, wherein both the CCI-779 and antimetabolite antineoplastic agent are administered weekly.

17. The method according to claim 16, where the administrations of CCI-779 and the antimetabolite antineoplastic agent are staggered uniformly.

18. The method according to claim 14, wherein the administration of the antimetabolite antineoplastic agent is immediately preceded by administration of a biochemical modifying agent.

19. The method according to claim 18, wherein said biochemical modifying agent is leucovorin.

20. The method according to claim 18, wherein said antimetabolite antineoplastic agent is 5-fluorouracil.
